(19) United States
(12) Patent Application Publication
(10) Pub. No.: US 2017/0303899 A1
(43) Pub. Date: Oct. 26, 2017

(54) ULTRASOUND ADAPTIVE POWER MANAGEMENT SYSTEMS AND METHODS

(71) Applicant: EchoNous, Inc., Redmond, WA (US)

(72) Inventor: Todd Willsie, Seattle, WA (US)

(21) Appl. No.: 15/498,000

(22) Filed: Apr. 26, 2017

Related U.S. Application Data
(60) Provisional application No. 62/327,636, filed on Apr. 26, 2016.

(51) Int. Cl. A61B 8/00

(52) U.S. Cl. CPC A61B 8/56; A61B 8/461; A61B 8/4254; A61B 8/4444; A61B 8/4483

(57) ABSTRACT

Systems and methods for dynamically managing power consumption in an ultrasound device are provided herein. A transducer in an ultrasound device may have transmit and receive elements for respectively transmitting and receiving ultrasound signals. In at least one embodiment, the method includes sensing a motion of the transducer by a motion sensor that is coupled to the transducer. An amount of power consumed by the ultrasound device is then reduced, based on the sensed motion of the transducer. Reducing an amount of power consumption may include adjusting one or more operational parameters of the ultrasound device, such as but not limited to reducing the display frame rate, the receive aperture, or the transmit amplitude, or by decoupling power to one or more components of the ultrasound device. Alternatively or in addition, power consumption may be reduced based on signals received from a capacitive sensor and/or a patient contact sensor.

### BACKGROUND

### Technical Field

The present application pertains to ultrasound systems, and more particularly to ultrasound systems and methods for adaptively managing power consumption based on a sensed motion of the ultrasound transducer.

### Description of the Related Art

Ultrasound imaging is useful as an imaging modality in a number of environments. For example, in the field of healthcare, internal structures of a patient's body may be imaged before, during or after a therapeutic intervention. A healthcare professional may hold a portable ultrasound probe, or transducer, in proximity to the patient and move the transducer as appropriate to visualize one or more target structures in a region of interest in the patient. A transducer may be placed on the surface of the body or, in some procedures, a transducer is inserted inside the patient's body. The healthcare professional coordinates the movement of the transducer so as to obtain a desired representation on a screen, such as a two-dimensional cross-section of a three-dimensional volume.

Ultrasound may also be used to measure functional aspects of a patient, such as organ movement and blood flow in the patient. Doppler measurements, for example, are effective in measuring the direction and speed of movement of a structure, such as a heart valve or blood cells flowing in a vessel, relative to the transducer. Doppler echocardiography is widely used for evaluating the cardiocirculatory system of patients with known or suspected cardiovascular disease.

For many years, ultrasound imaging was effectively confined to large equipment operating in a hospital environment. Recent technological advances, however, have produced smaller ultrasound systems that increasingly are deployed in frontline point-of-care environments, e.g., doctors' offices. Nevertheless, smaller ultrasound systems typically lack the power, thermal management, and processing capabilities of larger systems. This generally results in limited runtime of the ultrasound imaging components, lower image resolution, and fewer features or modes of operation.

### BRIEF SUMMARY

The present application, in part, addresses a desire for smaller ultrasound systems, having greater portability, lower cost, and ease of use for different modes of ultrasound imaging, while at the same time providing high quality measurements and effective power consumption management.

The performance of portable ultrasound devices may be limited by the available power storage, for example, by a power supply including one or more batteries. Since the amount of electrical power that may be delivered by such a power supply over a period of time may be limited, reducing the power consumption in a portable ultrasound device extends the life-cycle or charging cycle of the power supply, and thus allows the ultrasound device to be used for a longer period of time before replacing or recharging the batteries becomes necessary. Moreover, other benefits may be realized from reducing power consumption in a portable ultrasound device, such as advantageously reducing the amount of heat to be dissipated during operation. Reducing power consumption further allows for operating the ultrasound device for a longer period of time, while staying within regulatory limits with respect to the temperature of the ultrasound transducer during patient contact.

In a typical use case, the ultrasound system is not coupled to the body and producing a diagnostic image 100% of the time. This “non-imaging” time can include, for example, time spent applying ultrasound gel, moving the transducer to the patient, positioning the transducer to obtain the correct, desired view and confirming whether the image(s) captured are desirable.

The incorporation of motion sensing technology—such as, for example, accelerometers, gyroscopes and the like—in the ultrasound transducer can provide information about the motion of the transducer, and may be used to indicate the level of image quality that is possible at any given time (e.g., image quality may be reduced while the transducer is being moved). Information about the motion of the transducer (e.g., the acceleration or velocity of the transducer) can be used to control other system parameters in order to reduce the power consumption of the ultrasound device. Reducing power consumption in an ultrasound device, which may result in a lower quality ultrasound image, may be especially desirable or beneficial when the transducer motion already exceeds a predetermined threshold so as to reduce the likelihood of obtaining ultrasound images of higher quality or reliable diagnostic value. In such a case, after the motion of the transducer is reduced to a more acceptable or normal level (e.g., to a low enough level of motion that images of a desired quality may be obtained) then the power-related parameters of the system may be adjusted towards a normal operating level in order to obtain ultrasound images of a desired quality. Such capability in an ultrasound system may avoid power being wasted during an operating phase of the system where images are already likely to be unreliable or not of sufficient quality.

Other sensors may also be incorporated into the ultrasound device, which may be operatively coupled to the transducer or other portions of the ultrasound device, in order to provide information concerning the readiness of the system to capture images. For example, the ultrasound transducer may include a patient contact or pressure sensor positioned on an imaging surface of the transducer. The patient contact or pressure sensor may thus sense when the transducer is contacting, for example, a patient or a gel applied to the patient's skin. Power consumption may thus be reduced when the patient contact or pressure sensor senses that the transducer is not contacting the patient.

Similarly, the ultrasound transducer may include a capacitive sensor positioned to sense whether the transducer is being held, for example, by an operator of the ultrasound device. Power consumption may thus be reduced when the capacitive sensor senses that the transducer is not being held, and thus is not positioned to obtain ultrasound images of a desirable quality.

In at least one embodiment, a method is provided for dynamically managing power consumption in an ultrasound device having a transducer, the transducer including transmit and receive elements for respectively transmitting and receiving ultrasound signals. The method includes sensing a motion of the transducer by a motion sensor coupled to the transducer, and reducing an amount of power consumption by the ultrasound device based on the sensed motion of the transducer. Reducing an amount of power consumption may include adjusting one or more operational parameters of the ultrasound device, such as, for example, reducing a frame rate of the display, reducing a receive aperture of the transducer, reducing an amplitude of the ultrasound signals transmitted by the transducer or reducing the brightness of a display or otherwise reducing the power consumption required to deliver information to the user.

In another embodiment, the present disclosure provides a method for adaptively managing power consumption in an ultrasound device having a transducer. The method includes generating, by a motion sensor operatively coupled to the transducer, a motion sense signal indicating a motion of the transducer. The method further includes transmitting the motion sense signal to a power management controller, determining by the power management controller, based on the motion sense signal, whether the motion of the transducer exceeds a predetermined threshold level of motion, and reducing a level of power consumption by the ultrasound device if the motion of the transducer equals or exceeds the predetermined threshold level of motion.

In another embodiment, a handheld ultrasound transducer is provided that includes one or more first transducer elements, one or more second transducer elements and a motion sensor configured to sense one or more motions of the ultrasound transducer. The first transducer elements are arranged along an imaging surface of the ultrasound transducer and configured to transmit an ultrasound signal toward a target structure in a region of interest. The second transducer elements are arranged along the imaging surface of the ultrasound transducer and configured to receive echo signals returning from the target structure in response to transmission of the ultrasound signal.

In yet another embodiment, the present disclosure provides an ultrasound device that includes a handheld ultrasound transducer, processing circuitry, driving circuitry, a display and a power management controller. The handheld ultrasound transducer includes one or more first transducer elements arranged along an imaging surface of the ultrasound transducer and configured to transmit an ultrasound signal toward a target structure in a region of interest, one or more second transducer elements arranged along the imaging surface of the ultrasound transducer and configured to receive echo signals returning from the target structure in response to transmission of the ultrasound signal, and a motion sensor configured to sense a motion of the ultrasound transducer. The processing circuitry controls transmission of the ultrasound signal from the one or more first transducer elements. The driving circuitry is operatively coupled to the one or more first transducer elements and the processing circuitry, and the driving circuitry drives the transmission of the ultrasound signal by the one or more first transducer elements in response to a control signal received from the processing circuitry. The display is configured to display ultrasound images acquired by the ultrasound device, and the power management controller is coupled to the motion sensor and configured to reduce an amount of power consumption by the ultrasound device based on the sensed motion of the ultrasound transducer.

### BRIEF DESCRIPTION OF THE SEVERAL VIEWS OF THE DRAWINGS

FIG. 1 is a schematic illustration of an ultrasound imaging device, in accordance with one or more embodiments of the present disclosure.

FIG. 2 is a block diagram illustrating components of the ultrasound device 10, in accordance with one or more embodiments of the present disclosure.

FIG. 3 is a flow diagram illustrating a method for adaptively managing power consumption in an ultrasound device, in accordance with one or more embodiments of the present disclosure.

### DETAILED DESCRIPTION

A portable ultrasound device may include a power management module or controller configured to selectively enter the ultrasound device into one or more “low power” (i.e., reduced power) modes. A low power mode may include reducing or eliminating the power consumption of one or more components within the ultrasound device. For example, the low power mode may include electrically decoupling a power source to a transducer, a transducer transmit element, a transducer receive element, an ultrasound device display, driving circuitry, processing circuitry and/or any other electronic component in the ultrasound device in order to temporarily reduce the power consumption of the device.

Additionally, or alternatively, the low power mode may include reducing the power consumed by one or more electronic components within the ultrasound device. The power consumption of the ultrasound device may be reduced by dynamically adjusting system parameters such as, for example, the frame rate of the ultrasound device display (i.e., the rate of user display refresh), the receive aperture (i.e., the number of elements used for display generation) and the transmission amplitude (i.e., transmission power may be reduced) or the brightness of a user display.

The power management controller may include circuitry to dynamically adjust system parameters, and may further include circuitry (e.g., one or more switches or transistors) for coupling and decoupling power from a power supply to the various electronic components within the ultrasound device.

The low power mode may be initiated based on sense signals provided from one or more sensors, such as motion sensors operatively coupled to the transducer. For example, if a motion sensor senses that the ultrasound transducer is being moved too quickly to capture an ultrasound image of sufficient quality or reliability, the power management controller may enter the ultrasound device into a low power mode in order to save power until the period of undesirably rapid movement has concluded. That is, since the ultrasound device may not be able to capture suitable ultrasound images while the transducer is being moved too quickly, the power management controller may shut down or otherwise reduce the power consumed by various electronic components within the ultrasound device, such as the transducer elements during that period.

In one or more embodiments, the sensors may include a patient contact or pressure sensor positioned on an imaging surface of the transducer. Such a sensor may thus sense when the transducer is contacting, for example, a patient or a gel applied to the patient's skin. The power management controller may be coupled to the sensors, and thus may initiate the low power mode upon receiving a sense signal indicating that the transducer is not contacting the patient.

Similarly, the sensors may include a capacitive sensor positioned to sense whether the transducer is being held, for example, by an operator of the ultrasound device. The power management controller may thus initiate the low power mode upon receiving a sense signal indicating that the transducer is not being held, and thus is not positioned to obtain ultrasound images of a desirable quality.

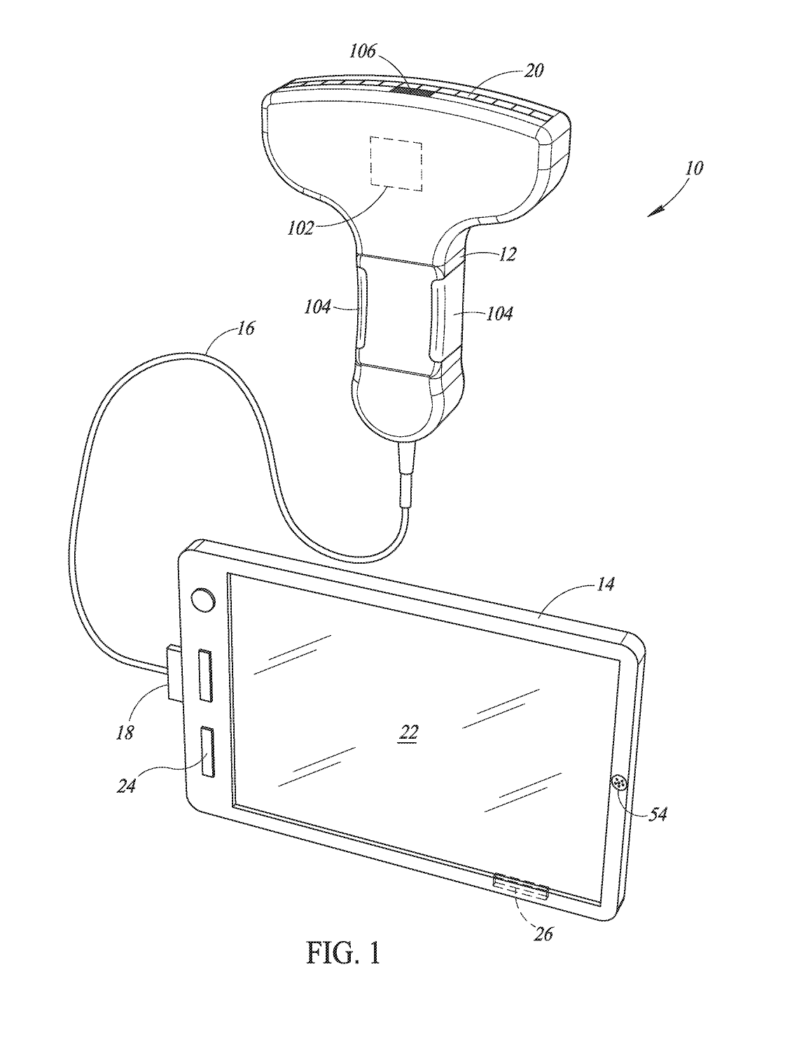

FIG. 1 is a schematic illustration of an ultrasound imaging device 10 (referred to herein as “ultrasound device” 10), in accordance with one or more embodiments of the present disclosure. The ultrasound device 10 includes an ultrasound transducer 12 that is electrically coupled to a computing device 14 by a cable 16. The cable 16 includes a connector 18 that detachably connects the transducer 12 to the computing device 14. As shown in FIG. 1, the ultrasound device 10 may be a portable ultrasound device, i.e., the transducer 12 may be connected to a portable computing device 14, such as a tablet computer, laptop, a hand-held device, or the like.

The transducer 12 is configured to transmit an ultrasound signal toward a target structure in a region of interest. The transducer 12 is further configured to receive echo signals returning from the target structure in response to transmission of the ultrasound signal. To that end, the transducer 12 includes transducer elements 20 that are capable of transmitting an ultrasound signal and receiving subsequent echo signals. In various embodiments, the transducer elements 20 may be arranged as elements of a phased array transducer. Suitable phased array transducers are known in the field of ultrasound technology.

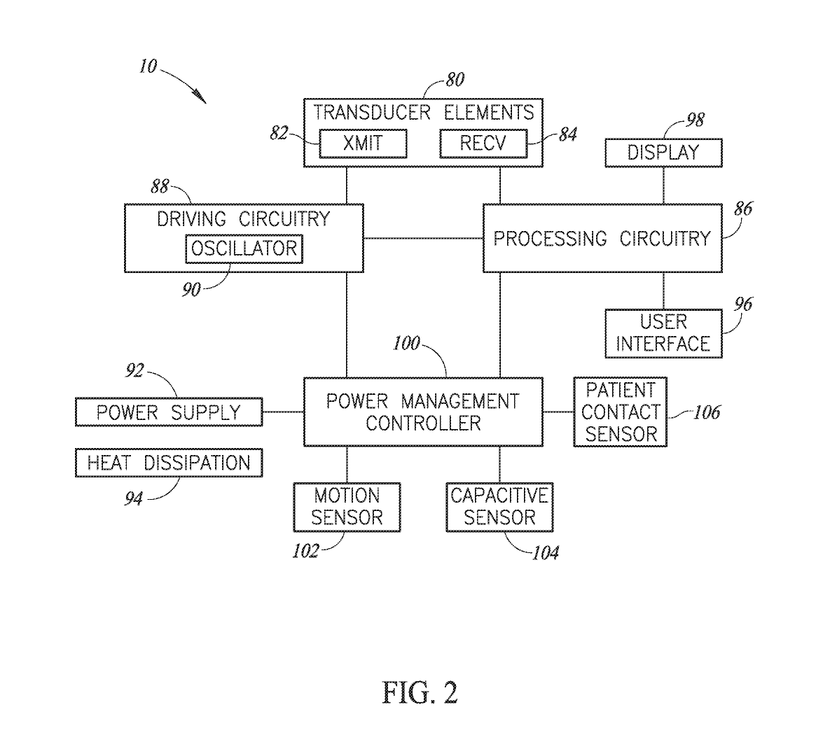

As will be described in greater detail in connection with FIG. 2, the ultrasound device 10 further includes processing circuitry and driving circuitry. In part, the processing circuitry controls the transmission of the ultrasound signal from the transducer elements 20. The driving circuitry is operatively coupled to the transducer elements 20 for driving the transmission of the ultrasound signal. The driving circuitry may drive the transmission of the ultrasound signal in response to a control signal received from the processing circuitry.

The ultrasound device 10 also includes a power supply that provides power to the driving circuitry for transmission of the ultrasound signal, for example, in a pulsed wave or a continuous wave mode of operation. Further, the ultrasound device 10 includes one or more sensors and a power management controller that dynamically adjusts power consumption in the ultrasound device 10, based on operating conditions such as motion of the transducer 12 and contact with a patient or with an operator of the ultrasound device 10, as will be described in further detail below. The sensors may include a motion sensor 102, a capacitive sensor 104 and a patient contact sensor 106.

The motion sensor 102 is included in the transducer 12 and may include, for example, one or more accelerometers or gyroscopes for sensing motion of the transducer 12. For example, the motion sensor 102 may be or include any of a piezoelectric, piezoresistive or capacitive accelerometer capable of sensing motion of the transducer 12, preferably in three dimensions.

One or more capacitive sensors 104 may further be included in the transducer 12 to sense whether the transducer 12 is being held by a user (e.g., an operator of the ultrasound device 10). As shown in FIG. 1, the capacitive sensor 104 may include one or more capacitive strips or elements positioned along the periphery of the transducer 12 such that, during normal operation of the ultrasound device 10, an operator's hand contacts the capacitive sensor 104 or is in close proximity with the capacitive sensor 104. While FIG. 1 shows capacitive sensors 104 for sensing human touch (e.g., when holding the transducer 12), it should be readily appreciated that any sensor capable of sensing physical contact (e.g., human touch) may be utilized in place of the capacitive sensors 104, including for example, one or more piezoresistive, piezoelectric, capacitive and elastoresistive sensors, as well as pressure sensors, force sensors and the like.

The patient contact sensor 106 may further be included in the transducer to sense whether the transducer 12 is contacting a physical object, such as a patient, during operation of the ultrasound device 10. The patient contact sensor 106 thus may be utilized to sense whether the transducer 12 is contacting a patient's skin or a gel applied to the patient's skin while operating the ultrasound device 10 to obtain ultrasound images. The patient contact sensor 106 may be positioned along an imaging surface of the transducer 12, such as a surface including the transducer elements 20, as shown in FIG. 1. The patient contact sensor 106 may be or include any tactile sensor, capacitive sensor, force sensor, pressure sensor or the like capable of sensing physical contact of a patient or gel applied to a patient with an imaging surface of the transducer 12.

The computing device 14 shown in FIG. 1 includes a display screen 22 and a user interface 24. The display screen 22 may use any type of display technology including, but not limited to, LED display technology. The display screen 22 is used to display one or more images generated from echo data obtained from the echo signals received in response to transmission of an ultrasound signal. In some embodiments, the display screen 22 may be a touch screen capable of receiving input from a user that touches the screen. In some embodiments, the user interface 24 may include one or more buttons, knobs, switches, and the like, capable of receiving input from a user of the ultrasound device 10.

The computing device 14 may further include one or more audio speakers 54 that may be used to generate audible representations of echo signals or other features derived from operation of the ultrasound device 10.

FIG. 2 is a block diagram illustrating components of the ultrasound device 10, including the ultrasound transducer 12 and the computing device 14. In FIG. 2, the ultrasound device 10 includes transducer elements 80 (e.g., transducer elements 20 shown in FIG. 1) configured for transmission of an ultrasound signal toward a target structure in a region of interest. The transducer elements 80 include one or more first transducer elements 82 that transmit the ultrasound signal and one or more second transducer elements 84 that receive echo signals returning from the target structure in response to transmission of the ultrasound signal. In some embodiments, some or all of the transducer elements 80 may act as first transducer elements 82 during a first period of time and as second transducer elements 84 during a second period of time that is different than the first period of time (i.e., the same transducer elements are usable to transmit the ultrasound signal and to receive echo signals at different times). In other embodiments, some or all of the first and second transducer elements 82, 84 may be different transducer elements, each configured for transmitting an ultrasound signal or receiving echo signals.

The ultrasound device 10 further includes processing circuitry 86 coupled to driving circuitry 88. In various embodiments, the processing circuitry 86 includes one or more programmed processors that operate in accordance with computer-executable instructions that, in response to execution, cause the programmed processor(s) to perform various actions. For example, the processing circuitry 86 may be configured to send one or more control signals to the driving circuitry 88 to control the transmission of an ultrasound signal by the ultrasound transducer 12.

The processing circuitry 86 is further coupled to a user interface 96 and a display 98. In at least one embodiment, the display 98 may comprise the screen 22 described with respect to FIG. 1, while the user interface 96 may comprise the interface elements 24 described with respect to FIG. 1.

The processing circuitry 86 may control a variety of operational parameters associated with the driving circuitry 86, the display 98 and the user interface 96.

The driving circuitry 88 may include an oscillator 90 that is used when generating an ultrasound signal to be transmitted by the one or more first transducer elements 82. The oscillator 90 is used by the driving circuitry 88 to generate and shape the ultrasound pulses that form the ultrasound signal.

The ultrasound device 10 includes a power supply 92 that is electrically coupled to component parts of the ultrasound device 10 through a power management controller 100. Such component parts may include, but are not limited to, the processing circuitry 86 and the driving circuitry 88. The power supply 92 provides power for operating the processing circuitry 86 and the driving circuitry 88. In particular, the power supply 92 provides power for generating the ultrasound signal by the driving circuitry 88 and transmitting the ultrasound signal, with stepped-up voltage as needed, by the one or more first transducer elements 82. The power provided by the power supply 92 also provides power for the driving circuitry 88 and the processing circuitry 86 when receiving echo signals via the one or more second transducer elements 84. The power supply 92 may further provide power for the display 98 and the user interface 96. The power supply 92 may be, for example, one or more batteries in which electrical energy is stored and which may be rechargeable.

During operation, components of the ultrasound probe 10, including but not limited to the power supply 92, generate heat that must be dissipated by the ultrasound probe 10. Accordingly, the ultrasound probe 10 may include one or more heat dissipation elements 94 configured to draw away and dissipate heat from the components of the ultrasound probe 10. For example, the heat dissipation elements 94 may include one or more thermal circuits that are thermally coupled to the components of the ultrasound device 10, including the power supply 92, and conduct heat toward a surface of the ultrasound device 10 for dissipation by convection to a user's hand or the surrounding environment.

The power management controller 100 controls the power drawn by the ultrasound device 10 based on sense signals provided from one or more of the motion sensor 102, the capacitive sensor 104 and the patient contact sensor 106. The power management controller 100 may control the power draw by adjusting operational parameters of the ultrasound device 10, and may further electrically decouple power from one or more components of the ultrasound device 10.

In one or more embodiments, the power management controller 100 may be included in, or executed by, the processing circuitry 86. For example, the power management controller 100 may be a module executed by one or more processors included in the processing circuitry 86. In other embodiments, the power management controller 100 may be configured with processing circuitry separate from the main processing circuitry 86 and may operate in cooperation with the processing circuitry 86. The processing circuitry of the power management controller 100 may be a programmed processor and/or an application specific integrated circuit configured to provide the power management functions described herein.

During operation of the ultrasound device 10, the motion sensor 102 senses motion of the transducer 12. The motion of the transducer 12 may indicate a level of ultrasound image quality which may be obtained at any given time. For example, obtaining high quality or clinically desirable ultrasound images may not be possible while moving the transducer 12 at a high rate of speed or acceleration in any direction. In contrast, by holding the transducer 12 still, at a proper position with respect to a target structure in a region of interest, a high or clinically desirable quality ultrasound image may be obtained. Thus, the sensed motion of the transducer 12 may be used as a proxy for, and may indicate, a level of ultrasound image quality which may be obtained at any instant in time.

The power management controller 100 receives a signal indicating motion of the transducer 12 from the motion sensor 102. Based on the sensed motion, the power management controller 100 may reduce the power consumption of the ultrasound device 10 by adjusting one or more operational parameters of the ultrasound device 10 or by adjusting the coupling of power to one or more components of the ultrasound device 10.

In one or more embodiments, the power management controller 100 may process the signal received from the motion sensor 102 to determine a motion of the transducer 12, and may compare the determined motion of the transducer 12 with a predetermined threshold motion. The motion of the transducer 12 may be reflected in terms of acceleration, velocity or other such parameter indicative of motion which may be detected by the motion sensor 102. The predetermined threshold motion may represent, for example, an acceleration or velocity at which (or above which) ultrasound images of a desired quality cannot be obtained. If the signal received from the motion sensor 102 indicates a motion (e.g., an acceleration or velocity) below the predetermined threshold motion, then the power management controller 100 will cause the components of the ultrasound device 10 (including, for example, the driving circuitry 88, the transducer elements 80, the display 98, the user interface 96 and/or the processing circuitry 86) to draw electrical power from the power supply 92 at a normal operating level. That is, the power management controller 100 will configure the ultrasound device 10 to operate in a normal or an “image acquisition” mode, as the motion of the transducer 12 is below a threshold level of motion and thus the ultrasound device 10 may obtain ultrasound images of a desired quality.

On the other hand, if the signal received from the motion sensor 102 indicates a motion (e.g., an acceleration or velocity) of the transducer 12 that equals or exceeds the predetermined threshold motion, then the power management controller 100 may determine that acquiring an ultrasound image of a desired quality is not possible. Accordingly, the power management controller 100 may configure the ultrasound device 10 to operate in a reduced or “low power” mode. That is, the power management controller 100 may adjust one or more operational parameters of the driving circuitry 88, the transducer elements 80, the display 98, the user interface 96, the processing circuitry 86 or any other electrical power consuming component of the ultrasound device 10, in order to reduce the power consumed by the ultrasound device 10 when the transducer 12 is moving too quickly or too fast to obtain ultrasound images of a desired quality. Alternatively, or in addition, the power management controller 100 may adjust the coupling of power to any of the electrical power consuming components to reduce the power consumption in the low power mode.

For example, in the low power mode, the power management controller 100 may reduce the power consumed by the ultrasound device 10 by reducing the frame rate of the display 98 (i.e., the rate at which the display 98 displays consecutive images acquired by the ultrasound device 10). Because ultrasound images of a desired quality cannot be obtained when the transducer 12 is moving at an acceleration or velocity that exceeds a predetermined threshold level of motion (e.g., acceleration or velocity), displaying the images acquired by the ultrasound device 10 at a normal frame rate while moving at such a rate may be of lesser importance than the amount of power consumed by the ultrasound device in such a scenario. Accordingly, the power management controller 100 may reduce the power consumption of the ultrasound device 10 by reducing the frame rate of the display 98.

Further, in the low power mode, the power management controller 100 may reduce the power consumed by the ultrasound device 10 by adjusting various parameters associated with the transducer elements 80 (e.g., transmit elements 82 and receive elements 84) and with beamforming or processing information received by the receive elements 84. For example, the power management controller 100 (or the driving circuitry 88 or processing circuitry 86, based on a control signal received from the power management controller 100) may dynamically reduce the receive aperture, or the number of transducer elements 80 used for image generation and display. By reducing the number of transducer elements 80 (e.g., the receive elements 84) used for display generation, the power required for low noise amplification and analog-to-digital conversion for forming the image is reduced.

Additionally, in the low power mode, the power management controller 100 may reduce the power used for transmission of an ultrasound signal (e.g., a transmit beam) by the transmit elements 82. For example, an amplitude of the transmit beam may be reduced, thereby reducing the power used by the ultrasound device 10 in the low power mode.

Any other operational parameters of the ultrasound device 10 may be adjusted in order to reduce the power consumed by the ultrasound device 10 when the power management controller 100 determines, based on the motion sensed by the motion sensor 102, that the transducer 12 is moving at a rate such that ultrasound images of a desired quality cannot be obtained.

A high rate of motion of the transducer 12 may indicate that the operator of the ultrasound device 10 is holding the transducer 12, but is moving the transducer 12, for example, to apply ultrasound gel to a patient or to position the transducer 12 in order to obtain a desired view. Since the operator is likely holding the transducer 12 in such a scenario, it may be preferable to decrease power consumption by entering a low power mode, as opposed to decoupling power to one or more components in the ultrasound device. However, in one or more embodiments, the power management controller 102 may electrically decouple one or more components of the ultrasound device 10 (e.g., the transducer elements 80, the driving circuitry 88, the display 98, etc.) from the power supply 92 based on motion sensed by the motion sensor 102. For example, the power management controller 100 may include one or more switches or transistors through which power from the power supply 92 is provided to the various components of the ultrasound device 10, and these switches or transistors may be opened if the motion of the transducer 12 exceeds a predetermined threshold, thereby decoupling power from the power supply 92 to those components.

In addition to the motion sensor 102, the power management controller 100 may reduce the power consumption of the ultrasound device 10 by adjusting one or more operational parameters of the ultrasound device 10 based on signals received from the capacitive sensor 104 and/or the patient contact sensor 106. For example, if the power management controller 100 receives a signal from the capacitive sensor 104 indicating that the transducer 12 is not being held, then the power management controller 100 may reduce the power consumed by one or more components of the ultrasound device 10, such as by reducing the frame rate, the receive aperture or the transmit amplitude.

Moreover, the power management controller 100 may decouple power from one or more components of the ultrasound device 10 based on a signal received from the capacitive sensor 104 indicating that the transducer 12 is not actively being held, e.g., by an operator. In such a case, for example, the power management controller 100 may decouple power from the power supply 92 to the driving circuitry 88, the transducer elements 80, the processing circuitry 86 or the display 98. Since the transducer 12 is not being held, it may be assumed that the operator is not actively trying to acquire ultrasound images, and as such, the power management controller 100 may effectively reduce power consumption by decoupling power from components while those components are not being used to obtain ultrasound images.

Similarly, if the power management controller 100 receives a signal from the patient contact sensor 106 indicating that the transducer 12 is not contacting a physical structure (e.g., a patient or gel applied to a patient), then the power management controller 100 may reduce the power consumed by one or more components of the ultrasound device 10, such as by reducing the frame rate, the receive aperture or the transmit amplitude. Additionally, or alternatively, the power management controller 100 may decouple power from one or more components of the ultrasound device 10 based on a signal received from the patient contact sensor 106 indicating that the transducer 12 is not positioned to obtain an ultrasound image (i.e., the transducer 12 is not contacting a structure or a subject containing the structure for imaging). In such a case, for example, the power management controller 100 may decouple or substantially reduce power from the power supply 92 to the driving circuitry 88, the transducer elements 80, the processing circuitry 86 or the display 98. Since the transducer 12 is not contacting a physical structure, it may be assumed that the operator is not actively trying to acquire ultrasound images, and thus the power management controller 100 may effectively reduce power consumption by decoupling power from components while those components are not being used to obtain ultrasound images.

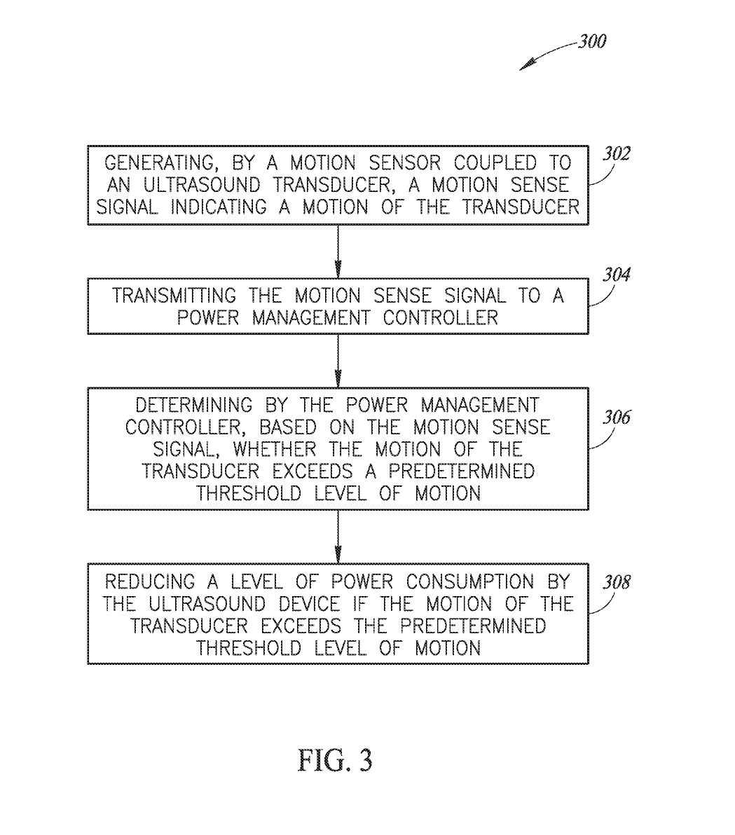

FIG. 3 is a flow diagram illustrating a method for adaptively managing power consumption in an ultrasound device 10 having a transducer 12, in accordance with one or more embodiments of the present disclosure. In at least one embodiment, the method 300 includes generating, by a motion sensor 102 coupled to the transducer 12, a motion sense signal indicating a motion of the transducer 12, as indicated at block 302. The motion sensor 102 may be, for example, one or more accelerometers or gyroscopes.

At block 304, the method 300 includes transmitting the motion sense signal to a power management controller 100. The power management controller 100 is coupled to a power supply 92 for supplying power to the ultrasound device 10, and is configured to adjust one or more operational parameters of one or more components in the ultrasound device 10.

At block 306, the method 300 includes determining by the power management controller 100, based on the motion sense signal, whether the motion of the transducer 12 exceeds a predetermined threshold level of motion. The predetermined threshold level of motion may be, for example, a predetermined threshold acceleration or velocity of the transducer 12 at or above which a desirable ultrasound image cannot be obtained by the transducer 12 or at least is not expected to be obtainable by the transducer 12.

At block 308, the method 300 includes reducing a level of power consumption by the ultrasound device 10 if the motion of the transducer 12 exceeds the predetermined threshold level of motion. The level of power consumption may be reduced by adjusting one or more operating parameters of the ultrasound transducer, including for example, reducing a frame rate of a display 98 of the ultrasound device 10, reducing a receive aperture of the transducer 12 and reducing an amplitude of the ultrasound signals transmitted by the transducer 12.

The ultrasound device 10 may further include one or more of a patient contact sensor 106 and a capacitive sensor 104. Thus, a method for adaptively managing power consumption in the ultrasound device 10 may further include generating, by the patient contact sensor 102, a contact sense signal indicating whether an imaging surface of the transducer 12 is contacting a physical structure; transmitting the contact sense signal to the power management controller 100; and reducing the level of power consumption by the ultrasound device 10 if the contact sense signal indicates that the imaging surface of the transducer 12 is not contacting a physical structure.

A method for adaptively managing power consumption in the ultrasound device 10 may further include generating, by the capacitive sensor 104, a capacitive sense signal indicating whether the transducer 12 is being held by an operator of the ultrasound device 10; transmitting the capacitive sense signal to the power management controller 100; and reducing the level of power consumption by the ultrasound device 10 if the capacitive sense signal indicates that the transducer 12 is not being held by the operator.

Reducing the level of power consumption by the ultrasound device may include electrically decoupling power to one or more of the transducer elements 80, the display 98, the driving circuitry 88 and the processing circuitry 86.

As may be appreciated by persons having ordinary skill in the art, aspects of the various embodiments described above can be combined to provide further embodiments. Aspects of the embodiments can also be modified, if necessary, to employ concepts of various patents, applications and publications in the relevant art to provide yet further embodiments.

These and other changes can be made to the embodiments in light of the above-detailed description. For example, in one or more embodiments a method, transducer and ultrasound device may be provided in which the power management controller 100 dynamically adjusts power consumption based on a signal received from a patient contact sensor 106, without a motion sensor 102. As such, the power management controller 100 may reduce power consumption based only on receiving a signal from the patient contact sensor 106 indicating that the transducer 12 is not contacting a physical structure (e.g., a patient or gel applied to a patient), such as by reducing the frame rate, the receive aperture or the transmit amplitude and/or by decoupling power to one or more components of the ultrasound device 10.

In yet another embodiment, a method, transducer and ultrasound device may be provided in which the power management controller 100 dynamically adjusts power consumption based on a signal received from a sensor configured to sense contact of a hand of an operator of the ultrasound device 10 with the transducer 12, e.g., capacitive sensor 104, without a motion sensor 102. As such, the power management controller 100 may reduce power consumption based only on receiving a signal from the capacitive sensor 104 indicating that the transducer 12 is not being held, such as by reducing the frame rate, the receive aperture or the transmit amplitude and/or by decoupling power to one or more components of the ultrasound device 10.

In still another embodiment, a method, transducer and ultrasound device may be provided in which the power management controller 100 dynamically adjusts power consumption based on signals received from any combination of the motion sensor 102, capacitive sensor 104 and/or patient contact sensor 106.

Additionally, in one or embodiments, a method, transducer and ultrasound device may be provided that include an “override” feature, which may be activated by a user, and which, when activated, prevents the system from entering a low power mode (i.e., in the override mode, the ultrasound device will continue to operate in a normal operational mode regardless of the parameters sensed by the motion sensor 102, capacitive sensor 104 and/or patient contact sensor 106). A user may activate the override feature, for example, via actuation of a physical button or switch, by adjusting a software setting (e.g., via a user interface provided through a display of the ultrasound device), or the like.

Further, in one or more embodiments, power may be conserved by turning off or otherwise reducing the power consumed by various features or elements of the ultrasound device once a certain level of battery power is detected. For example, the power management controller 100 may monitor a level of charge (e.g., a percentage of available battery power) of the batteries, and if the level of charge reaches or drops below a predetermined threshold (e.g., 10% of power, 20% of power, etc.), then the power management controller 100 may decouple power to and/or adjust one or more operational parameters associated with any feature or element of the ultrasound device as described herein in order to reduce power consumption. One such feature may include, for example, automatically uploading and/or downloading “deep learning” information from the cloud (e.g., downloading ultrasound image knowledge generated through a cloud-based artificial intelligence network and/or uploading acquired images to the cloud-based artificial intelligence network for further training). This feature is described, for example, in U.S. Provisional Patent Application No. 62/313,601 filed Mar. 25, 2016. The power management controller 100 may disable this feature (i.e., the ultrasound device will not download or upload information to the cloud-based artificial intelligence network) when the batteries are below a predetermined level of charge, and may cause the feature to remain disabled until the batteries are recharged to a level above the predetermined threshold level of charge. Any other features or elements of the ultrasound device may be disconnected from battery power and/or may have operational parameters that are adjusted in order to reduce power consumption when a level of charge of the batteries drops to or below the predetermined threshold. This may prevent, for example, a condition in which the ultrasound device loses power during an ultrasound imaging session.

In yet another embodiment, the ultrasound device may include a “sleep” mode feature that places the ultrasound device into a low or reduced power mode when the ultrasound transducer has not moved (e.g., as sensed by the motion sensors 102) for a period of time that exceeds a predetermined threshold (e.g., 10 seconds, 20 seconds, etc.). Additionally, or alternatively, the sleep mode may be initiated when the ultrasound transducer has not been held (e.g., as sensed by the capacitive sensor 104) for a period of time that exceeds a predetermined threshold. The ultrasound device may be returned to a normal (i.e., non-sleep mode) mode of operation upon detecting motion of the ultrasound transducer and/or upon detecting that the ultrasound transducer is being held.

In another embodiment, the power consumption of the ultrasound device may be reduced in a stepped manner based on an amount of time that the ultrasound transducer is motionless and/or is not being held (e.g., as sensed by the motion sensor 102 and/or the capacitive sensor 104). For example, the power management controller 100 may monitor an amount of time that the transducer is motionless and/or is not being held and may initiate a first low or reduced power mode after a first predetermined period of time (e.g., 10 seconds) has elapsed. The power management controller 100 may continue monitoring the amount of time that the transducer is motionless and/or is not being held and may initiate a second low or reduced power mode (e.g., by reducing power consumption of the ultrasound device even further than in the first low power mode) after a second predetermined period of time (e.g., 20 seconds) has elapsed. In the first low or reduced power mode, the power management controller 100 may, for example, adjust one or more operational parameters of the ultrasound device (e.g., by reducing the frame rate, the receive aperture, the transmit amplitude, the display brightness, etc.), while in the second low or reduced power mode, the power management controller 100 may further reduce power consumption by decoupling power to one or more components of the ultrasound device (e.g., the transducer elements 80, the display 98, the driving circuitry 88 and the processing circuitry 86).

In still another embodiment, the power management controller 100 may monitor a level of charge of the batteries, and may be configured to prevent the ultrasound device 10 from operating or otherwise being used to begin an ultrasound imaging session if the level of charge of the batteries is at or below a predetermined threshold level of charge. If the batteries are at or below the predetermined threshold level of charge, the ultrasound device may provide a message (e.g., a visual message provided via the display 22, an audible message or the like) informing the user to charge the ultrasound device 10 before beginning an ultrasound imaging session.

In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A method for dynamically managing power consumption in an ultrasound device having a transducer, the transducer including transmit and receive elements for respectively transmitting and receiving ultrasound signals, the method comprising:
sensing a motion of the transducer by a motion sensor coupled to the transducer; and
automatically reducing an amount of power consumption by the ultrasound device based on the sensed motion of the transducer.

2. The method of claim 1, wherein automatically reducing an amount of power consumption includes adjusting one or more operational parameters of the ultrasound device.

3. The method of claim 2, wherein the ultrasound device includes a display for displaying ultrasound images acquired by the ultrasound device, and wherein adjusting one or more operational parameters includes reducing a frame rate of the display.

4. The method of claim 2, wherein adjusting one or more operational parameters includes reducing a receive aperture of the transducer.

5. The method of claim 2, wherein adjusting one or more operational parameters includes reducing an amplitude of the ultrasound signals transmitted by the transducer.

6. A method for adaptively managing power consumption in an ultrasound device having a transducer, the method comprising:
generating, by a motion sensor coupled to the transducer, a motion sense signal indicating a motion of the transducer;
transmitting the motion sense signal to a power management controller;
determining by the power management controller, based on the motion sense signal, whether the motion of the transducer exceeds a predetermined threshold level of motion; and
automatically reducing a level of power consumption by the ultrasound device if the motion of the transducer exceeds the predetermined threshold level of motion.

7. The method of claim 6, wherein the predetermined threshold level of motion comprises at least one of a predetermined threshold acceleration and a predetermined threshold velocity of the transducer.

8. The method of claim 6, wherein the motion sensor includes at least one of an accelerometer and a gyroscope.

9. The method of claim 6, further comprising:
generating, by a patient contact sensor, a contact sense signal indicating whether an imaging surface of the transducer is contacting a physical structure;
transmitting the contact sense signal to the power management controller; and
automatically reducing the level of power consumption by the ultrasound device if the contact sense signal indicates that the imaging surface of the transducer is not contacting a physical structure.

10. The method of claim 9, wherein the patient contact sensor includes at least one of: a tactile sensor, a capacitive sensor, a force sensor and a pressure sensor.

11. The method of claim 9, wherein automatically reducing the level of power consumption by the ultrasound device if the contact sense signal indicates that the transducer is not contacting a physical structure includes electrically decoupling power to at least one of: a transducer element, a display, a driving circuit and a processing circuit.

12. The method of claim 6, further comprising:
generating, by a capacitive sensor, a capacitive sense signal indicating whether the transducer is being held by an operator of the ultrasound device;
transmitting the capacitive sense signal to the power management controller; and
automatically reducing the level of power consumption by the ultrasound device if the capacitive sense signal indicates that the transducer is not being held by an operator.

13. The method of claim 12, wherein automatically reducing the level of power consumption by the ultrasound device if the capacitive sense signal indicates that the transducer is not being held by an operator includes electrically decoupling power to at least one of: a transducer element, a display, a driving circuit and a processing circuit.

14. A handheld ultrasound transducer, comprising:
one or more first transducer elements configured to transmit an ultrasound signal toward a target structure in a region of interest;
one or more second transducer elements configured to receive echo signals returning from the target structure in response to transmission of the ultrasound signal;
a motion sensor configured to sense a motion of the ultrasound transducer; and
a power management controller configured to automatically reduce an amount of power consumption by the handheld ultrasound transducer based on a sensed motion of the ultrasound transducer.

15. The handheld ultrasound transducer of claim 14, further comprising:
a patient contact sensor arranged along an imaging surface of the ultrasound transducer, the patient contact sensor being configured to generate a contact sense signal indicating whether the imaging surface of the ultrasound transducer is contacting a physical structure.

16. The handheld ultrasound transducer of claim 14, further comprising:
a capacitive sensor configured to sense whether the transducer is being held by an operator of the ultrasound device.

17. An ultrasound device, comprising:
a handheld ultrasound transducer including:
one or more first transducer elements arranged along an imaging surface of the ultrasound transducer and configured to transmit an ultrasound signal toward a target structure in a region of interest,
one or more second transducer elements arranged along the imaging surface of the ultrasound transducer and configured to receive echo signals returning from the target structure in response to transmission of the ultrasound signal, and
a motion sensor configured to sense a motion of the ultrasound transducer;
processing circuitry that controls transmission of the ultrasound signal from the one or more first transducer elements;
driving circuitry operatively coupled to the one or more first transducer elements and the processing circuitry, wherein the driving circuitry drives the transmission of the ultrasound signal by the one or more first transducer elements in response to a control signal received from the processing circuitry;
a display configured to display ultrasound images acquired by the ultrasound device; and
a power management controller coupled to the motion sensor, the power management controller being configured to automatically reduce an amount of power consumption by the ultrasound device based on the sensed motion of the ultrasound transducer.

18. The ultrasound device of claim 17, wherein the handheld ultrasound transducer further includes a patient contact sensor arranged along an imaging surface of the ultrasound transducer, the patient contact sensor being configured to generate a contact sense signal indicating whether the imaging surface of the ultrasound transducer is contacting a physical structure,
wherein the power management controller is coupled to the patient contact sensor and configured to automatically reduce an amount of power consumption by the ultrasound device based on the contact sense signal.

19. The ultrasound device of claim 17, wherein the handheld ultrasound transducer further includes a capacitive sensor configured to sense whether the transducer is being held by an operator of the ultrasound device,
wherein the power management controller is coupled to the capacitive sensor and configured to automatically reduce an amount of power consumption by the ultrasound device based on an output of the capacitive sensor.

20. The ultrasound device of claim 17, wherein the power management controller is configured to automatically reduce an amount of power consumption by adjusting one or more operational parameters of the ultrasound device.
